(19) United States
(12) Patent Application Publication
(10) Pub. No.: US 2003/0220615 A1
(43) Pub. Date: Nov. 27, 2003

(54) Syringe, especially a pre-filled one-time-use syringe with a securely attachable finger rest

(76) Inventors: Arthur Fabian, Schaffhausen (CH); Ralf Holschumacher, Engelburg (CH); Bertram Eichhorn, Engelburg (CH); Walter Gygax, Teufen (CH)

Correspondence Address: Michael J. Striker, STRIKER, STRIKER & STENBY, 103 East Neck Road, Huntington, NY, 11743, US

(21) Appl. No.: 10/426,094

(22) Filed: Apr. 29, 2003

(30) Foreign Application Priority Data
May 4, 2002 (DE) 102 20 034.3

(51) Int. Cl. A61M 5/315

(52) U.S. Cl. 604/227

(57) ABSTRACT

The syringe, especially a pre-filled one-time-use syringe, has a syringe body, which is provided with a storage chamber for a liquid to be injected, and a piston with a piston rod so that the piston is axially slidable in the storage chamber. The syringe body has a finger rest and a stop member for limiting the axial motion of the piston at one end and the finger rest is in one-piece with the stop member, which includes at least one projecting element extending into the syringe body. The syringe has a syringe body with a radially outwardly extending flange section, which is formed for a forming-fitting and/or force-locking engagement with at least one catch region provided in the finger rest in order to securely hold the finger rest on the syringe body.

### BACKGROUND OF THE INVENTION

1. Field of the Invention

The present invention relates to a syringe, especially a pre-filled one-time-use syringe made of glass or plastic, with a syringe body which comprises a storage chamber for a liquid to be injected and a piston with a piston rod axially connected to it, so that the piston is axially slidable in the storage chamber, wherein the syringe body has a finger rest and a stop member for limiting the axial motion of the piston at one end and the finger rest is in one-piece with the stop member, which has at least one projection extending into the syringe body.

2. Description of the Related Art

In order to prevent an unintended withdrawal of the piston from the syringe body during storage, preparation and/or usage of a ready-made syringe, which is also called a one-time usage syringe, DE 100 36 829 A discloses a syringe in which a stop member is provided at one end opposite the needle, which is also called the “head end”. This stop member is formed by a circular narrowing of the syringe body of the known syringe.

A syringe with the features described in the section “Field of the Invention” above is known from DE 43 31 137 A. The known syringe has a syringe body, which has a reservoir for a liquid to be injected, a piston with piston rod received in it. The piston is arranged axially slidable by means of the piston rod within the injector body. To simplify its handling the known syringe has a stop member at its head end, which limits the axial movement of the piston and the piston rod connected with it. An embodiment of the known stop member is formed so that it has at least one projecting member extending into the injector body, against which the piston impacts during axial sliding in the direction of the head end of the syringe body. This presents an unintended or undesired withdrawal of the piston from the injector body.

This stop member in the known syringe, which is formed in one piece with the finger rest, is clamped laterally on the syringe body. In the assembled state the projecting member rests on the inner wall of the syringe body.

With this syringe according to the prior art there is however a danger that a sufficient attachment of the finger rest on the syringe body is not guaranteed because of the clipping of the stop member and the finger rest formed in one piece with it on the syringe body. Correspondingly during the injection process the syringe cannot be held securely by the respective user and accordingly can slip.

### SUMMARY OF THE INVENTION

It is an object of the present invention to provide a syringe or an injector of the above-described type, which is useable in an especially trouble-free manner.

It is an object of the present invention to provide a syringe or an injector of the above-described type, especially a pre-filled one-time-usage syringe, in which the finger rest is locked on the injector body so that the finger rest cannot detach from the syringe body during usage.

These objects and others, which will be made more apparent hereinafter, are attained in a syringe, especially a pre-filled one-time-use syringe made of glass or plastic, comprising a syringe body provided with a storage chamber for containing a liquid to be injected, a piston with a piston rod axially connected to it so that the piston is axially slidable in the storage chamber, a stop member for limiting the axial motion of the piston at one end and a finger rest attached to the syringe body at the one end, wherein the finger rest is in one-piece with the stop member, which has at least one projecting element extending into the syringe body.

According to the invention the syringe has a syringe body with a radially outwardly extending flange section, which is formed for a forming-fitting and/or a force-locking engagement with catch means, namely at least one catch region, provided in the finger rest, in order to securely hold the finger rest on the syringe body.

To establish the scope of the invention clearly the above wording “one end” of the syringe body, for the purposes of the present invention, means the end of the syringe body, which is also called the “head end” and which is the end that is opposite or remote from the needle or the needle attachment section of the syringe body.

The injector according to the invention has a series of advantages. First secure attachment of the finger rest with a simultaneously objection-free limiting of the axial motion of the piston within the syringe body is guaranteed by the structure according to the invention. This is due to the catch means or catch region provided in the finger rest, which can be engaged in a force-locking or form-fitting engagement with the flange section, which extends radially outward from the syringe body. Because of that the syringe according to the invention may be used and guided in an objection-free and reliable manner, when because of the outer, partially hectic conditions due to an emergency situation or sudden motions of the patient, an especially precise guidance of the syringe is required. This depends on the especially strong attachment of the syringe body with the at least one catch region and thus with the finger rest by the form-fitting and force-locking engagement of the catch region with the flange section. Because of that the finger rest cannot undesirably loosen from the syringe body or the finger rest cannot be displaced or shifted relative to the syringe body. Furthermore a finger rest of this type may be especially simply and rapidly mounted on the syringe body. It has the advantage that the axial motion of the piston and thus the connected piston rod is at the same time limited and terminated, so that an unwanted withdrawal or removal of the piston from the syringe body can be avoided. Thus the dangers associated with that removal can be permanently avoided. Subject to the requirement that, after filling the syringe body with the liquid to be injected, the finger rest and the stop member connected in one piece with it remain locked on the syringe body, the syringe body may be filled in an unobjectionable manner, since there is no undesirable narrowing or constriction of the syringe body at its head end. Because of the especially reliable locking of the finger rest on the syringe body, it is further possible to provide a large-area finger rest in the syringe according to the invention. The applications of the syringe according to the invention and simultaneously the reliablility of the syringe during usage are thus considerably increased.

In an especially appropriate embodiment according to the invention the flange section of the syringe body is formed as a circular flange at the end of the syringe body remote from the needle or needle attachment section. This circular flange extends around the entire circumference of the syringe body. Because of that the attachment of the finger rest is simplified and guaranteed so that the finger rest is still securely held during rotation of the finger rest relative to the syringe body.

In order to further simplify the attachment or application of the finger rest in the syringe according to the invention, in another embodiment of the syringe according to the invention the at least one catch region is at least one radially inwardly extending retaining region formed for receiving the flange section. Because of this preferred structure for the catch means or catch region it is possible to fix the finger rest with the syringe body especially securely, particularly by a form-fitting engagement of the at least one retaining region with the flange section. Understandably it is also possible to glue or melt the at least one radially inwardly extending retaining region with the flange section.

To provide additional fixing of the finger rest of the syringe or injector, in a further advantageous form of the above-described embodiment of the syringe according to the invention the catch means or region comprises two opposing retaining members, so that an especially reliable holding is provided with a minimum of material consumption. This structure provides a particularly reliable attachment of the finger rest to the syringe body, especially against unwanted shifting of the finger rest relative to the syringe body.

In regard to the form of the stop member for the piston that is formed in one piece with the finger rest any arbitrarily shaped stop member is suitable, which guarantees that the stop member projecting into the syringe body effectively prevents the piston from unwanted removal from the head end of the syringe body. Thus, for example, the stop member can be formed as a sleeve, which projects into the syringe body for a predetermined axial length. However it is especially preferred when the stop member has at least one pin-shaped projecting member, which projects from the head end of the syringe body into it so that this pin-shaped projecting member then limits and terminates the extent of the axial movement of the piston by contact with the piston during an axial motion of the piston in the direction of the head end.

Especially then, when the at least one projecting member rests on the interior wall of the injector body, one of these embodiments of the syringe according to the invention has a stop member, which effectively terminates the motion of the piston itself during a forceful upward motion of the piston in the direction of the head end of the syringe body without destroying the pin-shaped projecting member.

An additional advantageous form of the above-described embodiment of the syringe of the invention provides that the stop member comprises two pin-shaped projecting members, which are substantially identical and are arranged symmetrically around the circumference of the injector body so that they are arranged on opposite sides of the syringe body. This form of the stop member has the additional advantage that the stop member not only limits and terminates the axial motion of the piston in the direction of the head end of the syringe body, but at the same time prevents the finger rest from slipping transversely from the vertically positioned syringe body.

The embodiments of the syringe according to the invention in which two opposing pin-shaped projecting members are provided as the stop member are particularly advantageous from the standpoint of manufacturing and especially from the viewpoint of cost. The pin-shaped projecting members, as already describe above, preferably are arranged near the catch regions so that they extends axially into the syringe body and rest on the interior wall of the syringe body. Because of this feature, a slipping of the finger rest relative to the syringe body, even large-area finger rests, is effectively prevented.

For an especially suitable and very secure retaining of the finger rest on the syringe body, a preferred embodiment of the finger rest for the syringe according to the invention is a U-shape plate provided with two legs. Each leg has a respective catch region in it, which is preferably a radially inwardly projecting catch region, as is described in further detail in the detailed description of the preferred embodiment below. Each radially inwardly projecting catch region engages under the flange section of the syringe body. Because of the U-shape form of the finger rest axial slippage of it relative to the syringe body is prevented. If the stop element is a pin-shaped projection, which rests on the interior wall of the syringe body, and is supported there, in this embodiment of the syringe according to the invention even an extremely large finger rest is supported so that relative motion of the syringe body and the finger rest cannot occur. Moreover a finger rest formed in this way is very lightweight and easily attached.

In order to make the filling of the syringe body and the insertion of the piston and piston rod easy, a further embodiment of the syringe according to the invention provides that the preferably cylindrical syringe body has a widened interior diameter at the end in the vicinity of the finger rest.

Further advantageous features are described below and claimed in the appended dependent claims.

### BRIEF DESCRIPTION OF THE SEVERAL VIEWS OF THE DRAWING

The objects, features and advantages of the invention will now be illustrated in more detail with the aid of the following description of the preferred embodiment, with reference to the accompanying figures in which:

FIG. 1 is a schematic plan view of the top portion of a preferred embodiment of the syringe, but without the piston rod in it; and

FIG. 2 is a schematic cross-sectional view through the syringe shown in FIG. 1 taken along the section line A-A.

### DETAILED DESCRIPTION OF THE INVENTION

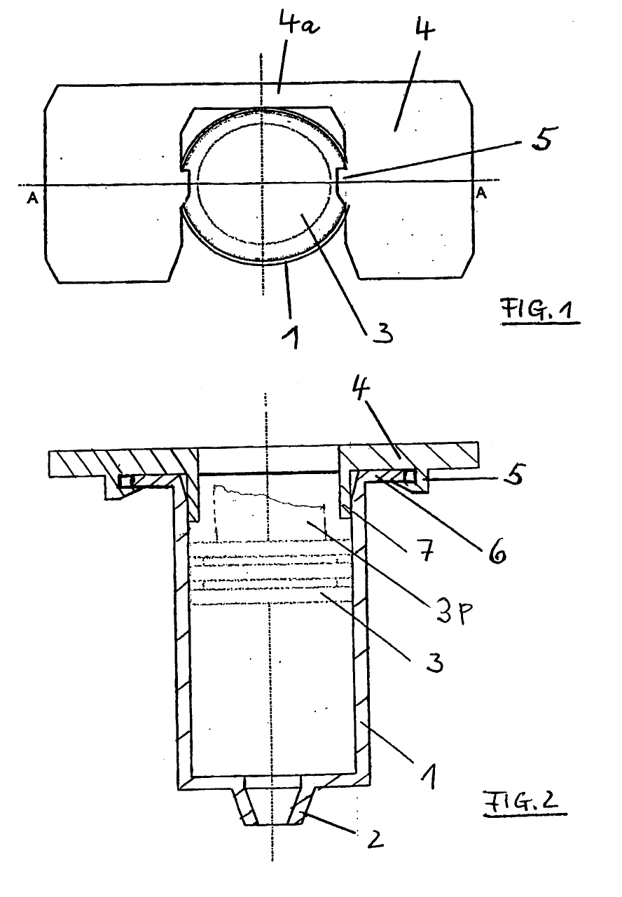

The same parts in the apparatus shown in FIGS. 1 and 2 are provided with the same reference numbers.

As can be best seen in FIG. 2, the syringe there has a syringe body 1 provided at its bottom end with an outlet section 2. A syringe needle, i.e. a hollow needle, can be attached or is attached in a conventional manner to this outlet section 2. A piston 3 is arranged within the injector body 1, which is slidable in an axial direction in the injector body 2 by means of a piston rod 3p shown cutaway in FIG. 2. The piston 3 is constructed in a conventional manner.

The one end of the syringe body 1 opposite from the outlet section 2 is provided with a finger rest 4, which has a hook-shaped retaining region 5. The hook-shaped retaining region 5 engages a circular flange 6 of the syringe body 1.

Furthermore the finger rest 4 has a stop member 7 formed as a pin-shaped or stud-shaped projecting member. The stop member 7 extends into the syringe body 1 so that the stop member 7 limits and halts axial travel of the piston 3 in the direction of the finger rest 4. The stop member 7 thus prevents unintended and uncontrolled removal of the piston 3 from the syringe body 1.

As seen best in FIG. 1, the finger rest 4 has a U-shape, when viewed from the top. It has two hook-shaped retaining regions 5 opposite from each other. Each retaining region 5 is assigned to or arranged near a respective pin-shaped projecting member or stop member 7.

In order to make insertion of the piston 3 into the interior of the injector or syringe body 1 easier and in order to simplify filling of the syringe body with liquid to be injected, a funnel-like widened portion of the syringe body 1 is provided in the vicinity of the finger rest at the one end. This widened portion has an interior diameter that is somewhat larger than the interior diameter of the remaining portion of the syringe body.

In order to attach the finger rest 4 at the end of the syringe body 1, first one retaining region 5 is arranged under the circular flange 6, so that the finger rest 4 can then be deformed in the vicinity of its thinnest or smallest section 4a. Because of that it is possible to guide the opposing stop members 7 into the syringe body 1. Then as a result of a gentle pressure and corresponding deformation the other retaining region 5 is moved away far enough until the appropriate hook-shaped retaining region 5 engages under the circular flange or flange section 6, whereby the finger support 4 is fixed on the syringe body 1 in an especially simple manner.

The disclosure in German Patent Application 102 20 034.3 of May 4, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in an injector, especially a pre-filled one-time-use injector, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A syringe comprising
a syringe body provided with a storage chamber for a liquid to be injected;
a piston with a piston rod axially connected to the piston so that the piston is axially slidable in the storage chamber;
a stop member for limiting axial motion of the piston, said stop member being arranged at one end of said syringe body; and
a finger rest attached to said one end of said syringe body;
wherein said stop member is in one piece with said finger rest and comprises at least one projecting element extending into said syringe body; and
wherein said syringe body has an radially outwardly extending flange section at said one end thereof and said finger rest includes at least one catch region formed for a form-fitting and/or a force-locking engagement with said radially outwardly extending flange section of the syringe body, in order to securely hold the finger rest on the syringe body.

2. The syringe as defined in claim 1, consisting of pre-filled one-time-use syringe made of glass or plastic.

3. The syringe as defined in claim 1, wherein said flange section is a circular flange.

4. The syringe as defined in claim 1, wherein said at least one catch region is a radially inwardly extending retaining region for receiving said flange section.

5. The syringe as defined in claim 1, wherein said at least one catch region comprises two radially inwardly extending retaining regions for receiving said flange section.

6. The syringe as defined in claim 1, wherein said stop member is at least one pin-shaped projecting member.

7. The syringe as defined in claim 6, wherein said at least one pin-shaped projecting member rests on an interior wall of said syringe body.

8. The syringe as defined in claim 5, wherein said stop member comprises two pin-shaped projecting members.

9. The syringe as defined in claim 7, wherein said two pin-shaped projecting members are identically formed and symmetrically arranged around a circumference of the syringe body.

10. The syringe as defined in claim 5, wherein said two radially inwardly extending retaining regions are opposite from each other, said stop member comprises two pin-shaped projecting members and each of said retaining regions is associated with one of said pin-shaped projecting members.

11. The syringe as defined in claim 1, wherein said at least one catch region consists of two retaining regions, said finger rest is a U-shape plate having two legs and each of said legs is provided with a respective one of said two retaining regions.

12. The syringe as defined in claim 1, wherein said syringe body has a widened portion at said one end in the vicinity of said finger rest so that an interior diameter of said syringe body is comparatively larger in said widened portion than that in a remaining portion of said syringe body outside of said syringe body.
